(19) United States
(12) Patent Application Publication
(10) Pub. No.: US 2016/0213732 A1
(43) Pub. Date: Jul. 28, 2016

(54) PILLOW USING ACORUS GRAMINEUS AND METHOD FOR MANUFACTURING SAME

(71) Applicant: Je Kyun LEE, Andong-si, Gyeongsangbuk-do (KR)

(72) Inventor: Je Kyun LEE, Andong-si, Gyeongsangbuk-do (KR)

(21) Appl. No.: 14/782,555

(22) PCT Filed: May 29, 2013
(86) PCT No.: PCT/KR2015/005421

§ 371 Date: Oct. 5, 2015

(30) Foreign Application Priority Data
Nov. 15, 2014 (KR) 10-2014-0159257

(51) Int. Cl. A61K 36/882; A61K 36/16; A61K 36/14; A61K 36/744; A61K 36/70; A61K 36/15; A61K 36/534; A61K 36/287; A01N 65/06; A01N 65/00; A01N 65/40; A01N 65/20; A01N 65/22; A01N 65/08; A01N 65/30; A01N 65/12; A61K 9/00; A01N 25/08; A47G 9/10; A61K 36/488

(52) U.S. Cl. CPC A61K 36/882; A61K 36/488; A61K 36/16; A61K 36/14; A61K 36/744; A61K 36/70; A61K 36/15; A61K 36/534; A61K 36/287; A01N 65/06; A01N 65/00; A01N 65/40; A01N 65/20; A01N 65/22; A01N 65/08; A01N 65/30; A01N 65/12; A61K 9/0087; A01N 25/08; A47G 9/10

(57) ABSTRACT

The present invention relates to a pillow using Acorus gramineus and a method for manufacturing the same, and more specifically, to a pillow containing Acorus gramineus and a method for manufacturing the same, wherein, when the pillow is manufactured using a plurality of natural materials in addition to Acorus gramineus, there can be presented processing methods for exhibiting the optimal medicinal effects of the natural materials; the human body can be protected from dust mites or the like through sterilizing and repellent effects; the shape of the pillow can be maintained; and sleep inducing and maintaining effects are excellent.

### TECHNICAL FIELD

The present invention relates to a pillow using Acorus gramineus and a method for manufacturing the same, and more specifically, to a pillow containing Acorus gramineus and a method for manufacturing the same, wherein, when the pillow is manufactured using a plurality of natural materials in addition to Acorus gramineus, there can be presented processing methods for exhibiting the optimal medicinal effects of the natural materials; the human body can be protected from dust mites or the like through sterilizing and repellent effects; the shape of the pillow can be maintained; and sleep inducing and maintaining effects are excellent.

### BACKGROUND ART

Sleep allows the restoration of homeostasis in human body tissues, the storage of energy, the control of body temperature, and the removal of inappropriate memories from sensory-overloaded brain (Pose et al., 2000), and the deprivation of sleep for a long period of time, that is, the loss of sleep for a long time results in deconstruction of self, hallucination, delusion, and the like (Kennaway et al., 1991).

The sleep has two physiological states, that is, a non-rapid eye movement (NREM) sleep state and a rapid eye movement (REM) sleep state (Friess et al., 1995, Kimura et al., 2000).

It is known that most physiological functions significantly deteriorate (stabilized pulses and regular breathing) in the NREM sleep state compared with the awakened state. The REM sleep state, which is similar to the awakened state, is characterized in that the brain functions and physiological functions are very actively expressed, and 90 minutes later falling asleep, the first REM sleep occurs following the NREM sleep 90 minutes (McGuinty et al., 1982).

One-third of adults may suffer from some form of sleep disorder during their lifetime. Insomnia is the sleep disorder that is most common and widely known. Insomnia refers to having difficulty in starting or maintaining sleep, and may happen temporarily or continuously (Maquet et al., 2003).

For the treatment of insomnia, causal treatment, drug treatment, environmental treatment, behavior therapy, and cognitive therapy are recommended. However, the drug treatment may cause drug dependency due to the use of a drug for a long period of time, and thus it is preferable to use the drug for a short time. Hence, improving the sleep environment is helpful in promoting or inducing sleep, leading to having a good sleep (Hauri et al., 1982, Weibel et al., 1996).

Here, the promotion or induction of sleep may be defined as inducing or promoting (helping) the introduction to a normal sleep state by improving the general condition in which it is hard to fall asleep.

Meanwhile, Acorus gramineus is used as a medicinal material, which is obtained by drying rhizomes of Acorus gramineus of the family Araceae. Acorus gramineus smells good due to aromaticity thereof, refreshes the mind to stabilize hearts and minds, treats forgetfulness, insomnia, ringing in ears, and is effective for a confused mind, weak hearing, dull eyes, and a heavy feeling in the head.

Hence, Korean Patent Publication Nos. 10-2006-0087785 and 10-2013-0082207 disclose the techniques of applying Acorus gramineus to a pillow. However, the patent publications disclose only the use of material through simple drying or the preparation into a powder or pill, but not a processing method for exhibiting the maximal medicinal efficacy. Moreover, the patent publications merely describe that the material has functionality by only general efficacy thereof, but do not present the reasons for the functionality, and thus it is not clear whether the material actually has a deep sleep effect.

### DETAILED DESCRIPTION OF THE INVENTION

### Technical Problem

Therefore, an aspect of the present invention is to provide processing methods suitable for a plurality of natural materials, respectively, so as to prolong the storage periods of the respective natural materials and produce the maximal medicinal effects of the respective natural materials when a pillow is manufactured using the natural materials besides Acorus gramineus.

Furthermore, another aspect of the present invention is to present clear criteria to manufacture a pill enabling a deep sleep by validating the sleep inducing and maintaining effects of the pillow, and to produce a medicinal effect on mental diseases (headache, neurodynia, insomnia, dementia, forgetfulness, and memory enhancement) through the deep sleep.

Furthermore, still another aspect of the present invention is to provide the promotion of health through repellent effects against dust mites, ticks, and photogens.

Furthermore, still another aspect of the present invention is to maintain the shape of the pillow regardless of the use of the pillow for a long time, thereby improving satisfaction of use.

### Technical Solution

In accordance with an aspect of the present invention, there is provided a pillow including, as pillow inner stuffing, 20-50 wt % of Acorus gramineus, 1-20 wt % of Pueraria thunbergiana, 1-20 wt % of ginkgo leaves, 1-10 wt % of Thuja orientalis, 1-10 wt % of Gardenia jasminoides, and 20-70 wt % of buckwheat chaff.

The pillow may further include any one or a mixture selected from pine needles, peppermint, and chrysanthemum, which are used alone or in a mixture thereof and of which each is further contained by 1-10 wt % on the basis of the total weight of the pillow inner stuffing regardless of the use type thereof.

In accordance with another aspect of the present invention, there is provided a method for manufacturing a pillow which includes, as pillow inner stuffing, 20-50 wt % of Acorus gramineus, 1-20 wt % of Pueraria thunbergiana, 1-20 wt % of ginkgo leaves, 1-10 wt % of Thuja orientalis, 1-10 wt % of Gardenia jasminoides, and 20-70 wt % of buckwheat chaff, wherein flowers, stems, and leaves of Acorus gramineus and Pueraria thunbergiana are freeze-dried; leaves of Thuja orientalis and the ginkgo leaves are collected in raw, and then subjected to a 9-times repeated steaming and drying process; and Gardenia jasminoides and the buckwheat chaff are dried.

The pillow may further include any one or a mixture selected from chrysanthemum, peppermint, and chrysanthemum, which are used alone or in a mixture thereof and of which each is further contained by 1-10 wt % on the basis of the total weight of the pillow inner stuffing regardless of the use type thereof, the chrysanthemum being subjected to a 9-times repeated steaming and drying process or a freeze-drying process, the pine needles and peppermint being subjected to a freeze-drying process.

Further, an inside cover of the pillow may be dyed with Gardenia jasminoides.

Hence, the pillow of the present invention can achieve a deep sleep, protect eyes, relieve the body fatigue, and refresh the head.

### Advantageous Effects

As set forth above, the present invention can provide various aids for the health of users through a deep sleep function allowing the users to sleep comfortably and functions for releasing stress due to brain activity and refreshing the head (dementia, forgetfulness, insomnia, headache, blood circulation, memory enhancement).

Further, the smells of materials included in the pillow of the present invention can refresh the head, and sustain the stability of the body and mind through sedative activity of the materials at ordinary times at home, even though the users are not in a sleep state.

Further, the materials are subjected to a freeze-drying or 9-times repeated steaming and drying process according to the characteristics thereof, thereby exhibiting the maximal medicinal efficacy; ginkgo and Gardenia jasminoides have a sterilizing effect or a pest repellent effect, thereby having a repellent effect against dust mites during the use of the pillow; and the buckwheat chaff maintains the shape and height of the pillow to prevent inconvenience, such as the lowering of the pillow, during the use of the pillow, thereby improving satisfaction to the pillow.

### BRIEF DESCRIPTION OF THE DRAWINGS

FIG. 1 shows a sleeping figure of a test animal.

FIG. 2 is a schematic view showing the sleeping time using the pillow inner stuffinging of the present invention.

### MODE FOR CARRYING OUT THE INVENTION

Hereinafter, examples of the present invention will be described with reference to the accompanying drawings.

The pillow according to the present invention includes, as inner stuffing, 20-50 wt % of Acorus gramineus, 1-20 wt % of Pueraria thunbergiana, 1-20 wt % of ginkgo leaves, 1-10 wt % of Thuja orientalis, 1-10 wt % of Gardenia jasminoides, and 20-70 wt % of buckwheat chaff. The pillow of the present invention further includes, besides the above materials, any one selected from pine needles, peppermint, and chrysanthemum, which may be used alone or in a mixture thereof and of which each is further contained by 1-10 wt % on the basis of the total weight of pillow inner stuffing regardless of the use type thereof.

Here, Acorus gramineus is washed, and then subjected to washing and fine cutting, followed by freeze-drying for 4 days, thereby maintaining the natural smell and shape thereof. The leaves of Acorus gramineus have a pungent smell.

In addition, Pueraria thunbergiana opens up the pores to discharge sweat, has special efficacy for alcoholic poisoning or thirst, and treats various diseases, including diabetes, edema, glycermia, hypertension, and angina. In order to keep the smell and shape of Pueraria thunbergiana, flowers, stems, and leaves of Pueraria thunbergiana are cut, cleanly washed, dried in a freeze-drier for 4 days in a freeze-drying manner, and then taken out for storage.

Gardenia jasminoides is a medicinal material that reduces a fever and calms a user down, and acts as a pest repellent. Gardenia jasminoides is dried and pulverized before being stored.

Thuja orientalis leaves exhibit a hemostatic action by narrowing the blood vessels and accelerating blood coagulation; performs an anti-inflammatory action, a bacteria suppressing action, and an antiviral action; and is used for preventing hair loss and prematurely graying of hairs. Thuja orientalis leaves are stored after only recently grown green leaves are cut, subjected to a 9-times repeated steaming and drying process using 2 mal of water in a pot over medium heat for 1 to 10 hours, and dried for 3 days.

The ginkgo leaves have insecticidal and disinfecting ingredients. When the ginkgo leaves are green in August and September before the ginkgo leaves fall, the ginkgo leaves are dried in a cool, shaded space, and then put in the pillow, thereby producing a dust mite preventing effect. The ginkgo leaves are helpful in concentration and brain health, and promote blood circulation. Especially, flavonoid contained in the ginkgo leaves is an effective antioxidant ingredient and prevents hair loss. The ginkgo leaves are stored after only recently grown green leaves are cut, subjected to a 9-times repeated steaming and drying process in a pot, and dried for 3 days.

The menthol ingredient contained in the peppermint has a unique flavor, and thus is widely used for toothpaste, flavoring, cookies, drinking water, and the like; is effective for headaches; and has an ant repellent action. Such peppermint is stored after the whole plant is dried and then cut.

With respect to chrysanthemums, flower buds are cut in fall, and in order to maintain the flavor and leaves in a natural state, the cut flower buds are cleanly washed, and then dried in a freeze-drier for 4 days in a freeze-drying manner before storage thereof, or subjected to a 9-times repeated steaming and drying process before use thereof.

The buckwheat chaff cools the head due to a cooling property thereof; has effects in the prevention of forgetfulness, dementia, and insomnia; has effects of preventing hypertension and cerebral hemorrhaging; and is dried in a natural drying manner or by a hot-water drier before storage.

In addition, pine needles have effects of promoting hairgrowth and stabilizing five internal organs. Raw leaves of the pine needles are collected, and then freeze-dried before storage.

Meanwhile, with respect to the pillow of the present invention, an inside cover, into which all the pillow inner stuffing is inserted, may be inserted and sealed by a zipper in an outside cover, which is used to improve the beauty of the pillow and absorb sweat. Alternatively, the inside cover is formed as a double layer, of which an inner layer is filled with the other inner stuffing except for buckwheat and an outer layer is filled with buckwheat, and the layers of the inside cover may be dyed with Gardenia jasminoides.

The effects of the pillow of the present invention will be described with reference to the following examples.

I. Materials and Method

1. Test on Simple Sleep Promotion Function

For evaluation of the sleep promotion function, the efficacy of Acorus gramineus was evaluated by a total of two tests.

First, a sleep promotion comparison test was conducted using normal bedding and Acorus gramineus pillow inner stuffing of the present invention. Second, a test was conducted by checking, as indicators, physiological responses, such as behavior change and eyelid closure, which occurred when sleep was promoted after the administration of caffeine as a chemical stimulus.

1) Animals

In the study, 6-week old male ICR mice (Samtako Co, Gyeonggi, Korea) were purchased with an average weight of 30.2±0.8 g for the use. After acclimatization with a basic feed for 3 days, the mice were grouped into two groups, and tested three times for three weeks, with free access to food and water. The animals' room was maintained at a temperature of 23° C., humidity of 50-60%, ventilation of 10-15 times/hour, and an illumination of 200-300 Lux under a 12-h light/dark cycle with no stress conditions. The animal test was performed following guidelines of the Animal Research Ethics Committee at Wonkwang University (Approval No. WKU). The mice were given free access to a fixed raw material mixed feed and purified water filtered and sterilized by a filter and an ultraviolet sterilizer.

The test animals are grouped into two groups, and the simple sleep promotion function was measured. Both of the two groups were given free access to food and water. Group I used normal bedding (Samtako, Korea), and group II used Acorus gramineus pillow inner stuffing. In addition, a fasting plate is placed in a cage, and the animal was kept from eating the bedding and the Acorus gramineus pillow inner stuffing. The normal bedding and the Acorus gramineus pillow inner bedding were exchanged with new ones every day at 10 AM, and a constant amount of 40 g was used for stuffing for each exchange (Table 2).

2) Design of Animal Test

After 6-week old growing ICR mice were received, the mice were acclimatized with normal food for 3 days, and then housed in a cage with a fasting plate. The mice were grouped into a total of two groups, that is, a group using normal bedding (I, n=6) and a group using Acorus gramineus pillow inner stuffing as bedding (II, n=6), and then the test on the simple sleep promotion effect was conducted. The group using normal bedding and the group using Acorus gramineus pillow inner stuffing as bedding were weighed using an electronic scale on the test start day and the test end day (10 AM). The number of mice constituting each group was selected by the minimum number so as to identify the statistical significance following the principle of 3R.

3) Test Material

The Acorus gramineus pillow inner stuffing of the present invention is composed of 20-50 wt % of roots, leaves, and flowers of Acorus gramineus, 1-20 wt % of flowers, stems, and leaves of Pueraria thunbergiana, 1-20 wt % of ginkgo leaves, 1-10 wt % of leaves and stems of Thuja orientalis, 1-10 wt % of Gardenia jasminoides, 1-10 wt % of pine needles, 20-70 wt % of buckwheat chaff, and 1-10 wt % of Chrysanthemum zawadskii as chrysanthemum.

4) Measurement on Simple Sleeping Time

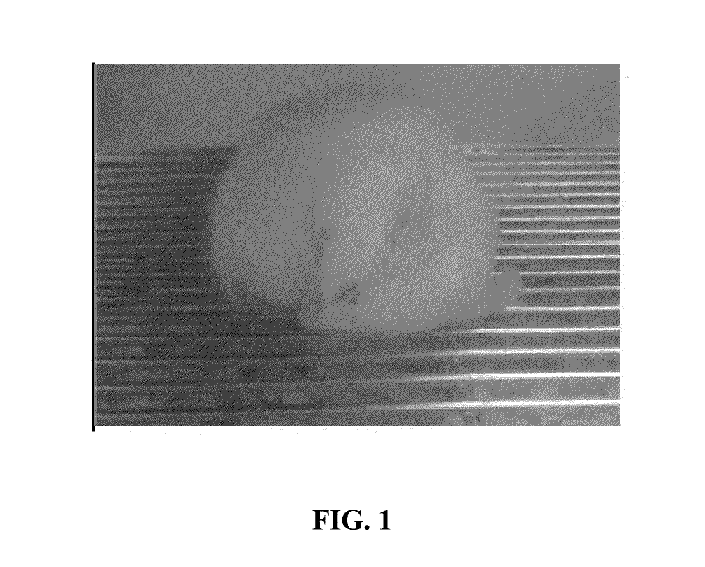

The sleeping time was measured on the basis of righting reflex in 6-week old ICR mice. In order to measure the degree at which the animal feels sleepy or the degree at which the animal tends to enter the resting state, the sleeping time was measured from a time point at which the animal curled up and slept, as shown in FIG. 1, by the method proposed by Jansen.

2. Test on Sleep Promotion in Sleep Disorder Induction

1) Animals

In the study, 6-week old male ICR mice (Samtako Co, Gyeonggi, Korea) were purchased with an average weight of 28.4±0.78 g for the use. After acclimatization with basic feed for 3 days, the mice were grouped into two, and tested three times for three weeks, with free access to feed and water. The animals' room was maintained at a temperature of 23° C., humidity of 50-60%, ventilation of 10-15 times/hour, and an illumination of 200-300 Lux under a 12-h light/dark cycle with no stress conditions. The animal test was performed following guidelines of the Animal Research Ethics Committee at Wonkwang University (Approval No. WKU). The mice were given free access to a fixed raw material mixed feed and purified water filtered and sterilized by a filter and an ultraviolet sterilizer.

Test animals were grouped into four groups, and the sleep promotion function was measured in the presence of sleep disturbance factors. All of the four groups were given free access to food and water. Here, group I used normal bedding (Samtako, Korea) with an administration of a saline solution; group II used normal bedding (Samtako, Korea) with an administration of caffeine 1 mg/kg; group III used Acorus gramineus pillow inner stuffing with an administration of 1 mg/kg; and group IV used Acorus gramineus pillow inner stuffing with an administration of a saline solution. In addition, a fasting plate is placed in a cage, and the animal was kept from eating the bedding and the Acorus gramineus pillow inner stuffing. The normal bedding and the Acorus gramineus pillow inner bedding were exchanged with new ones every day at 10 AM, and a constant amount of 40 g was used for stuffing for each exchange.

2) Design of Animal Test

After 6-week old growing ICR mice were received, the mice were acclimatized with normal food for 3 days, and then housed in a cage with a fasting plate. The mice were grouped into a total of four groups, that is, a group using normal bedding (Samtako, Korea) with an administration of a saline solution (I, n=6); a group using normal bedding (Samtako, Korea) with an administration of caffeine 1 mg/kg (II, n=6); a group using Acorus gramineus pillow inner stuffing with an administration of 1 mg/kg (III, n=6); and a group using Acorus gramineus pillow inner stuffing with an administration of a saline solution (IV, n=6), and then a test on the sleep promotion function was conducted in the presence of sleep disturbance factors. The group using normal bedding and the group using Acorus gramineus (Acorus gramineus pillow inner stuffing) as bedding were weighed using an electronic scale on the test start day and the test end day (10 AM). The number of mice constituting each group was selected by the minimum number so as to identify the statistical significance following the principle of 3R.

3) Sleep Disorder Induction in Animals

The test was started 30 minutes after oral administration of caffeine 1 mg/kg based on Velluci (1984) (Takahashi et al., 1992; Kaneko et al., 1996; Liao et al., 2002; Kim et al., 2003; Choi et al., 2003).

II. Results

1. Test Results on Simple Sleep Promotion Function

In the two groups using normal bedding and using Acorus gramineus pillow inner stuffing as bedding, respectively, the group using normal bedding showed that one of six mice was sleep-induced within 45.3±1.3 minutes and the sleeping time was 169±8.5 minutes, and the group using Acorus gramineus pillow inner stuffing showed that five of six mice was sleep-induced within 28.2±3.2 minutes, and the sleeping time was 202.33±12.56 minutes.

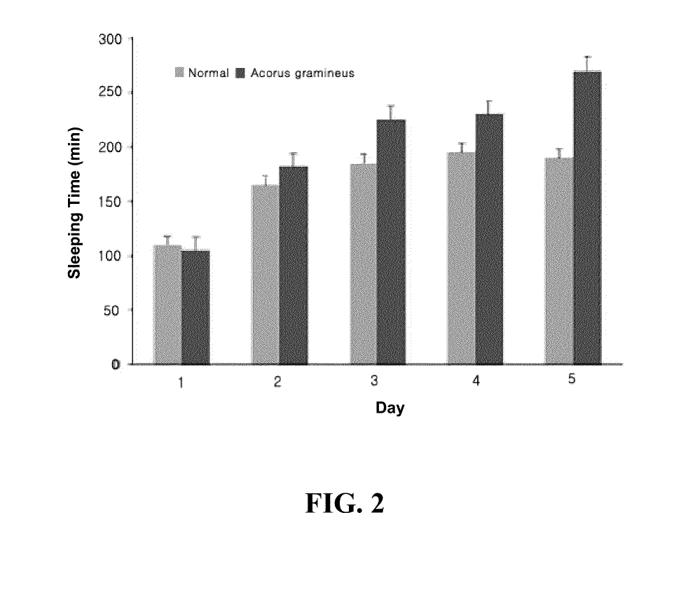

Therefore, it was verified that the sleep inducing time was shortened in the use of Acorus gramineus pillow inner stuffing as bedding rather than in the use of normal bedding, and it could be seen that the sleeping time was also longer in the use of Acorus gramineus pillow inner stuffing as bedding rather than in the use of normal bedding, as shown in FIG. 2.

2. Test Results on Sleep Promotion in Sleep Disorder Induction

The sleeping time was 139.17±26.73 minutes in the group using normal bedding with an administration of a saline solution (I); 47.50±17.50 minutes in the group using normal bedding with an administration of caffeine 1 mg/kg (II); 76.67±48.37 minutes in the group using Acorus gramineus pillow inner stuffing with an administration of 1 mg/kg (III); and 180.00±42.06 minutes in the group using Acorus gramineus pillow inner stuffing with an administration of a saline solution, and thus it can be seen that the sleeping time was longer when compared with the normal food.

The above test results confirmed that the sleep inducing time was shortened and the sleeping time was prolonged in the groups Acorus gramineus pillow inner stuffing rather than in the groups using normal bedding. Therefore, it is determined that the use of the pillow of the present invention shortens the sleep inducing time and prolongs the sleeping time, allowing a deep sleep.

Meanwhile, the sensory evaluation using the pillow of the present invention was conducted.

That is, the Acorus gramineus pillow of the present invention was tested on 120 persons aged from their 20s to 70s with 20 persons for each group, using a 4-point scoring method (e.g., 4: very satisfactory, 3: satisfactory, 2: unsatisfactory, 1: very unsatisfactory) of the sleeping depth, pillow aroma, sense of fatigue (headache), and overall preference, as shown in table 2 below.

TABLE 1

| | Depth of | Pillow | Sense of fatigue | Overall |
|---|---|---|---|---|
| | deep sleep | aroma | (headache) | preference |
| 20s | 3.5 | 3.3 | 3.6 | 3.5 |
| 30s | 3.3 | 3.4 | 3.7 | 3.5 |
| 40s | 3.8 | 3.6 | 3.8 | 3.6 |
| 50s | 3.7 | 3.6 | 3.8 | 3.7 |
| 60s | 3.6 | 3.7 | 3.6 | 3.6 |
| 70s | 3.2 | 3.3 | 3.5 | 3.3 |

As a result, it can be seen that the pillow of the present invention has excellent overall preference since the subjects have a deep sleep, no repulsion to the aroma, and an improved sense of fatigue (headache) after the deep sleep.

1. A pillow comprising, as pillow inner stuffing, 20-50 wt % of Acorus gramineus, 1-20 wt % of Pueraria thunbergiana, 1-20 wt % of ginkgo leaves, 1-10 wt % of Thuja orientalis, 1-10 wt % of Gardenia jasminoides, and 20-70 wt % of buckwheat chaff.

2. The pillow of claim 1, further comprising any one or a mixture selected from pine needles, peppermint, and chrysanthemum, which are used alone or in a mixture thereof and of which each is further contained by 1-10 wt % on the basis of the total weight of the pillow inner stuffing regardless of the use type thereof.

3. A method for manufacturing a pillow which comprises, as pillow inner stuffing, 20-50 wt % of Acorus gramineus, 1-20 wt % of Pueraria thunbergiana, 1-20 wt % of ginkgo leaves, 1-10 wt % of Thuja orientalis, 1-10 wt % of Gardenia jasminoides, and 20-70 wt % of buckwheat chaff, wherein flowers, stems, and leaves of Acorus gramineus and Pueraria thunbergiana are freeze-dried; leaves of Thuja orientalis and the ginkgo leaves are collected in raw, and then subjected to a 9-times repeated steaming and drying process; and Gardenia jasminoides and the buckwheat chaff are dried.

4. The method of claim 3, wherein the pillow further comprises any one or a mixture selected from chrysanthemum, peppermint, and chrysanthemum, which are used alone or in a mixture thereof and of which each is further contained by 1-10 wt % on the basis of the total weight of the pillow inner stuffing regardless of the use type thereof, the chrysanthemum being subjected to a 9-times repeated steaming and drying process or a freeze-drying process, the pine needles and peppermint being subjected to a freeze-drying process.

5. The method of claim 3, wherein an inside cover of the pillow is dyed with Gardenia jasminoides.
